(19) United States
(12) Patent Application Publication
(10) Pub. No.: US 2015/0099914 A1
(43) Pub. Date: Apr. 9, 2015

(54) PROCESS FOR THE AROMATIZATION OF A METHANE-CONTAINING GAS STREAM

(71) Applicant: SHELL OIL COMPANY, Houston, TX (US)

(72) Inventors: Juan Mirabel Garza, Richmond, TX (US); Daniel Edward Gerwien, Sugar Land, TX (US); David Morris Hamilton JR., Sugar Land, TX (US); Larry Lanier Marshall, Houston, TX (US); Waleed Yousef Musallam, Houston, TX (US); Anand Nilekar, Houston, TX (US); Peter Tanev, Katy, TX (US); Lizbeth Olivia Cisneros Trevino, Katy, TX (US)

(21) Appl. No.: 14/395,821

(22) PCT Filed: Apr. 23, 2013
(86) PCT No.: PCT/US13/37692

§ 371 Date: Oct. 21, 2014

Related U.S. Application Data
(60) Provisional application No. 61/636,906, filed on Apr. 23, 2012.

(51) Int. Cl. C07C 2/76

(52) U.S. Cl. CPC C07C 2/76; C07C 2529/48
USPC 585/417

(57) ABSTRACT

A process for the aromatization of a methane-containing gas stream comprising: contacting the methane-containing gas stream in a reactor with a fluidized bed comprising an aromatization catalyst and a hydrogen acceptor under methane-containing gas aromatization conditions to produce a product stream comprising aromatics and hydrogen wherein the hydrogen is, at least in part, bound by the hydrogen acceptor in the reaction zone and removed from the product and the reaction zone.

### FIELD OF THE INVENTION

This invention relates to a process for the aromatization of a methane-containing gas stream to form aromatics and hydrogen in a reactor containing both catalyst and hydrogen acceptor particles in a fluidized bed state wherein the removal of hydrogen from the reaction zone is accomplished insitu by the hydrogen acceptor.

### BACKGROUND

The aromatic hydrocarbons (specifically benzene, toluene and xylenes) are the main high-octane bearing components of the gasoline pool and important petrochemical building blocks used to produce high value chemicals and a variety of consumer products, such as styrene, phenol, polymers, plastics, medicines, and others. Since the late 1930's, aromatics are primarily produced by upgrading of oil-derived feedstocks via catalytic reforming or cracking of heavy naphthas. However, occasional severe oil shortages and price spikes result in severe aromatics shortages and price spikes. Therefore, there is a need to develop new and independent from oil, commercial routes to produce high value aromatics from highly abundant and inexpensive hydrocarbon feedstocks such as methane or stranded natural gas (typically containing about 80-90% vol. methane).

There are enormous proven reserves of stranded natural gas around the world. According to some estimates, the world reserves of natural gas are at least equal to those of oil. However, unlike the oil reserves that are primarily concentrated in a few oil-rich countries and are extensively utilized, upgraded and monetized, the natural gas reserves are much more broadly distributed around the world and significantly underutilized. Many developing countries that have significant natural gas reserves lack the proper infrastructure to exploit them and convert or upgrade them to higher value products. Quite often, in such situations, natural gas is flared to the atmosphere and wasted. Because of the above reasons, there is enormous economic incentive to develop new technologies that can efficiently convert methane or natural gas to higher value chemical products, specifically aromatics.

In 1993, Wang et al., (Catal. Lett. 1993, 21, 35-41), discovered a direct, non-oxidative route to partially convert methane to benzene by contacting methane with a catalyst containing 2.0% wt. molybdenum on H-ZSM-5 zeolite support at atmospheric pressure and a temperature of 700° C. Since Wang's discovery, numerous academic and industrial research groups have become active in this area and have contributed to further developing various aspects of the direct, non-oxidative methane to benzene catalyst and process technology. Many catalyst formulations have been prepared and tested and various reactor and process conditions and schemes have been explored.

Despite these efforts, a direct, non-oxidative methane aromatization catalyst and process cannot yet be commercialized. Some important challenges that need to be overcome to commercialize this process include: (i) the low, as dictated by thermodynamic equilibrium, per pass conversion and benzene yield (for example, 10% wt. and 6% wt., respectively at 700° C.); (ii) the fact that the reaction is favored by high temperature and low pressure; (iii) the need to separate the produced aromatics and hydrogen from unreacted (mainly methane) hydrocarbon off gas and (iv) the rapid coke formation and deposition on the catalyst surface and corresponding relatively fast catalyst deactivation. Among these challenges, overcoming the thermodynamic equilibrium limitations and significantly improving the conversion and benzene yield per pass has the potential to enable the commercialization of an efficient, direct, non-oxidative methane containing gas aromatization process.

The methane aromatization reaction can be described as follows:

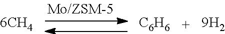

According to the reaction, 6 molecules of methane are required to generate a molecule of benzene. It is also apparent that, the generation of a molecule of benzene is accompanied by the generation of 9 molecules of hydrogen. Simple thermodynamic calculations revealed and experimental data have confirmed that, the methane aromatization at atmospheric pressure is equilibrium limited to about 10 and 20% wt. at reaction temperature of 700 or 800° C., respectively. In addition, experimental data showed that the above conversion levels correspond to about 6 and 11.5% wt. benzene yield at 700 and 800° C., respectively. The generation of 9 molecules of hydrogen per molecule of benzene during the reaction leads to significant volume expansion that suppresses the reaction to proceed to the right, i.e. it suppresses methane conversion and formation of reaction products, i.e. benzene yield. The aforementioned low per pass conversions and benzene yields are not very attractive to provide an economic justification for scale-up and commercialization of methane containing gas aromatization process.

Therefore, there is a need to develop an improved direct, non-oxidative methane containing gas stream aromatization process that provides for significantly higher (than allowed by the thermodynamic equilibrium) conversion and benzene yields per pass by implementing an insitu hydrogen removal from the reaction zone.

### SUMMARY OF THE INVENTION

The invention provides a process for the aromatization of a methane-containing gas stream comprising: contacting the methane-containing gas stream in a reactor with a fluidized bed comprising an aromatization catalyst and a hydrogen acceptor under methane-containing gas aromatization conditions to produce a product stream comprising aromatics and hydrogen wherein the hydrogen is, at least in part, bound by the hydrogen acceptor in the reaction zone and removed from the product and reaction zone.

The invention further provides unique process schemes for recycling and regenerating the catalyst and hydrogen acceptor particles wherein both catalyst and hydrogen acceptor particles are continuously withdrawn from the reaction zone of the reactor, fed to a regeneration vessel or vessels to be regenerated and returned back into the reactor for continuous (uninterrupted) production of aromatics and hydrogen. The aforementioned insitu hydrogen removal in a fluidized bed state allows for overcoming the thermodynamic equilibrium limitations and for shifting the reaction equilibrium to the right. This results in significantly higher and economically more attractive methane-containing gas stream conversion and benzene yields per pass relative to the case without hydrogen removal in the reaction zone.

### BRIEF DESCRIPTION OF THE DRAWINGS

FIG. 1 shows a schematic diagram of an embodiment of the invention: aromatization reactor with catalyst and hydrogen acceptor particles intermixed in a fluidized bed.

FIG. 2 shows a schematic diagram of an embodiment of the invention: co-regeneration of both hydrogen acceptor and catalyst particles in a single vessel.

FIG. 3 shows a schematic diagram of another embodiment of the invention: regeneration of hydrogen acceptor and catalyst particles in separate vessels.

### DETAILED DESCRIPTION

The conversion of a methane-containing gas stream to aromatics is typically carried out in a reactor comprising a catalyst, which is active in the conversion of the methane-containing gas stream to aromatics. The methane-containing gas stream that is fed to the reactor comprises more than 50% vol. methane, preferably more than 70% vol. methane and more preferably of from 75% vol. to 100% vol. methane. The balance of the methane-containing gas may be other alkanes, for example, ethane, propane and butane. The methane-containing gas stream may be natural gas which is a naturally occurring hydrocarbon gas mixture consisting primarily of methane, with up to about 30% vol. concentration of other hydrocarbons (usually mainly ethane and propane) as well as small amounts of other impurities such as carbon dioxide, nitrogen and others.

The conversion of a methane-containing gas stream is carried out at a gas hourly space velocity of from 100 to 60000 h⁻¹, a pressure of from 0.5 to 10 bar and a temperature or from 500 to 900° C. More preferably, the conversion is carried out at gas hourly space velocity of from 300 to 30000 h⁻¹, a pressure of from 0.5 to 5 bar and a temperature of from 700 to 875° C. Even more preferably, the conversion is carried out at gas hourly space velocity of from 500 to 10000 h⁻¹, a pressure of from 0.5 to 3 bar and a temperature of from 700 to 850° C. Various co-feeds such as CO, CO₂ or hydrogen or mixtures thereof that react with coke precursors or prevent their formation during methane aromatization could be added at levels of <10% vol. to the methane-containing feed to improve the stability, performance or regenerability of the catalyst. The methane-containing gas aromatization is then carried out until conversion falls to values that are lower than those that are economically acceptable. At this point, the aromatization catalyst has to be regenerated to restore its aromatization activity to a level similar to its original activity. Following the regeneration, the catalyst is again contacted with a methane-containing gas stream in the reaction zone of the aromatization reactor under aromatization conditions for continuous production of aromatics.

Any catalyst suitable for methane-containing gas stream aromatization may be used in the process of this invention. The catalyst typically comprises one or more active metals deposited on an inorganic oxide support and optionally comprises promoters or other beneficial compounds. The active metal or metals, promoters, compounds as well as the inorganic support all contribute to the overall aromatization activity, mechanical strength and performance of the aromatization catalyst.

The active metal(s) component of the catalyst may be any metal that exhibits catalytic activity when contacted with a gas stream comprising methane under methane-containing gas aromatization conditions. The active metal may be selected from the group consisting of: vanadium, chromium, manganese, zinc, iron, cobalt, nickel, copper, gallium, germanium, niobium, molybdenum, ruthenium, rhodium, silver, tantalum, tungsten, rhenium, platinum and lead and mixtures thereof. The active metal is preferably molybdenum.

The promoter or promoters may be any element or elements that, when added in a certain preferred amount and by a certain preferred method of addition during catalyst synthesis, improve the performance of the catalyst in the methane-containing gas stream aromatization reaction.

The inorganic oxide support can be any support that, when combined with the active metal or metals and optionally the promoter or promoters contributes to the overall catalyst performance exhibited in the methane aromatization reaction. The support has to be suitable for treating or impregnating with the active metal compound or solution thereof and a promoter compound or solution thereof. The inorganic support preferably has a well-developed porous structure with sufficiently high surface area and pore volume and suitable for aromatization surface acidity. The inorganic oxide support may be selected from the group consisting of: zeolites, non-zeolitic molecular sieves, silica, alumina, zirconia, titania, yttria, ceria, rare earth metal oxides and mixtures thereof. The inorganic oxide support of this invention contains zeolite as the primary component. The zeolite is selected from the group consisting of ZSM-5, ZSM-22, ZSM-8, ZSM-11, ZSM-12 or ZSM-35 zeolite structure types. The zeolite is preferably a ZSM-5 zeolite. The ZSM-5 zeolite further may have a SiO₂/Al₂O₃ ratio of 10 to 100. Preferably, the SiO₂/Al₂O₃ ratio of the zeolite is in the range of 20-50. Even more preferably the SiO₂/Al₂O₃ ratio is from 20 to 40 and most preferably about 30. The support may optionally contain about 15-70% wt of a binder that binds the zeolite powder particles together and allows for shaping of the catalyst in the desired form and for achieving the desired high catalyst mechanical strength necessary for operation in a commercial aromatization reactor. More preferably the support contains from 15-30% wt. binder. The binder is selected from the group consisting of silica, alumina, zirconia, titania, yttria, ceria, rare earth oxides or mixtures thereof.

The final shaped catalyst could be in the form of cylindrical pellets, rings or spheres. The preferred catalyst shape of this invention (for fluidized bed reactor operation) is spherical. The spherical catalyst of this invention could be prepared by any method known to those skilled in the art. Preferably, the spherical catalyst of this invention is prepared via spray drying of zeolite containing sols of appropriate concentration and composition. The zeolite containing sol may optionally contain binder. The spherical catalyst has predominant particle size or diameter that makes it suitable for fluidization. The spherical particle diameter of the catalyst of this invention is preferably selected to be in the range of 20-500 microns. More preferably, the spherical catalyst of this invention has particle diameter in the range of 50-200 microns.

The hydrogen acceptor used in this reaction can be any metal-containing alloy or a compound that has the ability, when subjected to aromatization operating conditions, to selectively accept or react with hydrogen to form a sufficiently strong hydrogen-acceptor bond. The hydrogen acceptor preferably reversibly binds the hydrogen in such a way that during operation in the fluidized bed reactor the hydrogen is strongly bound to the acceptor under the methane-containing gas stream aromatization conditions. In addition, the hydrogen acceptor is preferably able to release the hydrogen when transported to the regeneration section where it is subjected to a different set of (regeneration) conditions that favor release of the previously bound hydrogen and regeneration of the hydrogen acceptor.

Suitable hydrogen acceptors include: Ti, Zr, V, Nb, Hf, Co, Mg, La, Pd, Ni, Fe, Cu, Ag, Cr, Th as well as other transition metals, elements or compounds or mixtures thereof. The hydrogen acceptor may comprise metals that exhibit magnetic properties, such as for example Fe, Co or Ni or various ferro-, para- or dimagnetic alloys of these metals. One or more hydrogen acceptors that exhibit appropriate particle sizes and mass for fluidized bed aromatization operation may be used in the reaction zone to achieve the desired degree of hydrogen separation and removal.

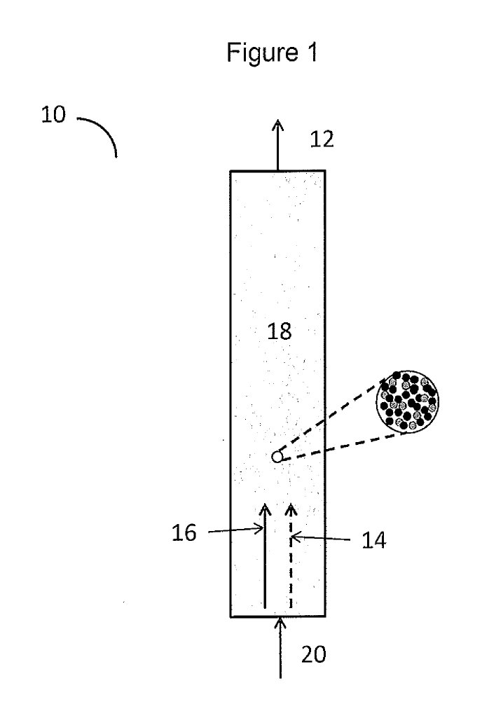

The aromatization reaction of this invention is carried out in a fluidized bed reactor. To enable this, suitably shaped and sufficiently robust catalyst and hydrogen acceptor particles that are able to sustain the rigors of high severity fluidized-bed operation under aromatization reaction conditions are prepared and used for the reaction. According to the present invention, the use of the catalyst and hydrogen acceptor in a fluidized bed reactor and configuration provides several important advantages over the prior art. The most significant advantage of the process of this invention is that it provides for insitu removal of hydrogen from the reaction zone and as a consequence, significant increase of both methane-containing gas stream conversion and benzene yield per pass to values that are significantly higher relative to these dictated by the methane aromatization reaction equilibrium. This is enabled by mixing and placing the catalyst and hydrogen acceptor particles in a fluidized-bed state in the reaction zone or the aromatization reactor (see FIG. 1). In FIG. 1, a fluidized bed reactor 10 comprises a mixture of catalyst and hydrogen acceptor particles in the fluidized bed 18. The methane-containing gas stream, the catalyst and hydrogen acceptors are introduced via one or more inlets (20) and the products, unreacted gases, catalyst and hydrogen acceptor are removed from the bed via one or more outlets 12. The feed and products flow upward in the direction of arrow 16. The catalyst and hydrogen acceptor are introduced upwardly in the direction of arrow 14 (although the catalyst and hydrogen acceptor then form a fluidized bed)

The mixing of both types of particles in a fluidized bed state provides for the quick removal of the produced hydrogen from the reaction zone and for shifting the aromatization reaction equilibrium toward greater methane-containing gas conversion and benzene yields per pass. Another advantage of the present invention is that it allows, under fluidized bed operating conditions, for volume expansion of the hydrogen acceptor particles during the process of binding of hydrogen to take place. Hydrogen acceptors undergo significant volume expansion in the process of binding hydrogen and at some point in the process the hydrogen acceptor will bind so much hydrogen that it reaches its maximum hydrogen binding capacity. If the acceptor were used in a fixed bed reactor configuration it would expand and agglomerate in the confined bed volume. This would cause agglomeration of the hydrogen acceptor particles, plugging and significant reactor pressure drop, and suppression of the aromatization reaction.

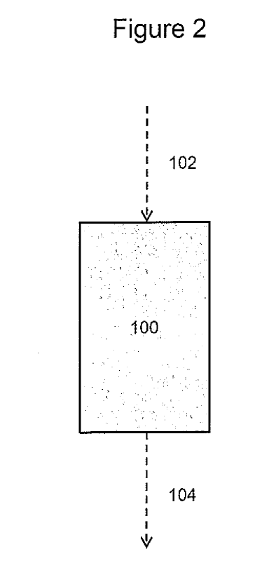

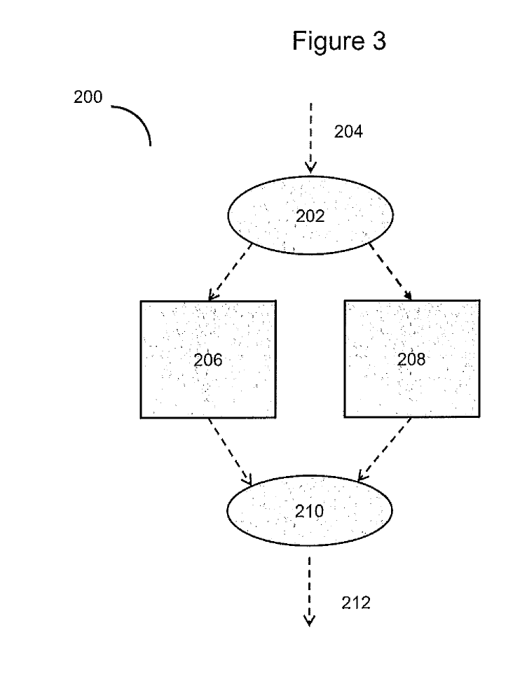

Yet another advantage of the present invention is that, the particle shapes, sizes and mass of both hydrogen acceptor and catalyst particles are designed and selected in such a way so that they could be co-fluidized in the reactor to form the desired fluidized bed. Also, the invention provides for two or more different by chemical formula and/or physical properties hydrogen acceptors to be simultaneously used with the catalyst in the fluidized bed reactor to achieve the desired degree of hydrogen separation from the aromatization reaction zone. Another important advantage of the process of this invention is that it provides for the catalyst and the hydrogen acceptor particles to be simultaneously and continuously withdrawn from the reaction zone or the reactor, regenerated in separate vessel or vessels according to one of the schemes illustrated in FIGS. 2 and 3 and then continuously returned back to the reactor for continuous aromatics and hydrogen production. The hydrogen acceptor and catalyst regeneration could be accomplished either simultaneously or stepwise in the same vessel as illustrated in FIG. 2 or separately in separate vessels as illustrated in FIG. 3. This later operation scheme provides for maximum flexibility to accomplish the hydrogen release or regeneration of the acceptor and catalyst under different and suitable for the purpose set of operating conditions. The regeneration of catalyst and hydrogen acceptor could be accomplished in fixed, moving or fluidized bed reactor vessels schematically shown in FIGS. 2 and 3.

In FIG. 2, regenerator vessel 100 is used to regenerate the catalyst and regenerate the hydrogen acceptor. The catalyst and hydrogen acceptor particles are introduced via inlet 102 and are then removed via outlet 104. Hydrogen removed from the hydrogen acceptor and gases produced by catalyst regeneration are removed from the regenerator via one or more outlets (not shown).

In FIG. 3, regenerator system 200 comprises a separation step 202 to separate the catalyst from the hydrogen acceptor that is fed from the reactor via line 204. The catalyst is fed to catalyst regeneration vessel 206, and the hydrogen acceptor is fed to hydrogen acceptor regeneration vessel 208. The catalyst and hydrogen acceptor are then mixed back together in mixing step 210 and then fed back to the reactor via line 212.

In the case of separate regeneration (see FIG. 3), the hydrogen acceptor particles could be separated from the catalyst on the basis of (but not limited to) differences in mass, particle size, density or on the basis of difference in magnetic properties between the acceptor and the catalyst particles. In the later case, the hydrogen acceptor of this invention could be selected from the group of materials exhibiting fero-, para-or diamagnetic properties and comprising Fe, Co or Ni. It is well known that, the methane-containing gas aromatization catalysts form coke during the reaction. Accumulation of coke on the surface of the catalyst gradually covers the active aromatization sites of the catalyst resulting in gradual reduction of its activity.

Therefore, the coked catalyst has to be removed at certain carefully chosen frequencies from the reaction zone of the aromatization reactor and regenerated in one of the regeneration vessels depicted in FIGS. 2 and 3. The regeneration of the catalyst can be carried out by any method known to those skilled in the art. For example, two possible regeneration methods are hot hydrogen stripping and oxidative burning at temperatures sufficient to remove the coke from the surface of the catalyst. If hot hydrogen stripping is used to regenerate the catalyst, then at least a portion of the hydrogen used for the catalyst regeneration may come from the hydrogen released from the hydrogen acceptor. Additionally, fresh hydrogen may be fed to the catalyst regeneration vessel as needed to properly supplement the hydrogen released from the hydrogen acceptor and to complete the catalyst regeneration. If the regeneration is carried out in the same vessel (see FIG. 2), then the hydrogen removed from the hydrogen acceptor in-situ could at least partially hydrogen strip and regenerate the catalyst. If the regeneration is carried out in different vessels (see FIG. 3) the operating conditions of each vessel could be selected and maintained to favor the regeneration of the catalyst or the hydrogen acceptor. Hydrogen removed from the hydrogen acceptor could then again be used to at least partially hydrogen strip and regenerate the catalyst.

Yet another important advantage of the process of this invention over the prior art is that it provides for the release of the hydrogen that is bound to the hydrogen acceptor when the saturated acceptor is subjected to a specific set of conditions in the regeneration vessel(s). Furthermore, the released hydrogen could be utilized to regenerate the catalyst or subjected to any other suitable chemical use or monetized to improve the overall aromatization process economics.

Another important advantage of the present invention is that it allows for different regeneration conditions to be used in the different regeneration vessel or vessels to optimize and minimize the regeneration time required for the catalyst and hydrogen acceptor and to improve performance in the aromatization reaction.

The aforementioned advantages of the process of the present invention provide for an efficient removal of hydrogen from the reaction zone of methane-containing gas aromatization reactor operating in fluidized bed mode and for shifting the reaction equilibrium towards higher methane-containing gas stream conversion and benzene yields per pass. Therefore, the present invention has the potential to allow for the commercialization of an economically attractive direct, non-oxidative methane-containing gas stream aromatization process.

1. A process for the aromatization of a methane-containing gas stream comprising: contacting the methane-containing gas stream in a reactor with a fluidized bed comprising an aromatization catalyst and a hydrogen acceptor under methane-containing gas aromatization conditions to produce a product stream comprising aromatics and hydrogen wherein the hydrogen is, at least in part, bound by the hydrogen acceptor in the reaction zone and removed from the product and the reaction zone.

2. The process of claim 1 wherein the methane-containing gas stream conversion and corresponding benzene yield per pass are higher than the conversion and yield obtained with the same aromatization catalyst and under the same methane-containing gas aromatization conditions, but in the absence of a hydrogen acceptor in the reaction zone of the aromatization reactor.

3. The process of claim 1 wherein the methane-containing gas stream also comprises lower alkanes selected from the group consisting of ethane, propane and butane.

4. The process of claim 1 wherein the methane-containing gas stream comprises carbon dioxide.

5. The process of claim 1 wherein the methane-containing gas stream comprises at least 60% vol. methane.

6. The process of claim 1 wherein the aromatization catalyst comprises a zeolite selected from the group consisting of ZSM-5, ZSM-22, ZSM-8, ZSM-11, ZSM-12 or ZSM-35.

7. The process of claim 1 wherein the aromatization catalyst comprises a metal selected from the group consisting of vanadium, chromium, manganese, zinc, iron, cobalt, nickel, copper, gallium, germanium, niobium, molybdenum, ruthenium, rhodium, silver, tantalum, tungsten, rhenium, platinum and lead and mixtures thereof.

8. The process of claim 1 wherein the hydrogen acceptor comprises a metal or metals that are capable of selectively binding hydrogen under the methane-containing gas aromatization conditions in the reaction zone of a fluidized bed reactor.

9. The process of claim 1 wherein the hydrogen acceptor comprises a metal selected from the group consisting of Ti, Zr, V, Nb, Hf, Co, Mg, La, Pd, Ni, Fe, Cu, Ag, Cr, Th and other transition metals and compounds or mixtures thereof.

10. The process of claim 1 wherein the methane aromatization conditions comprise a temperature in the range of from 500° C. to 900° C.

11. The process of claim 1 further comprising continuously regenerating the catalyst to remove coke formed during the reaction and continuously regenerating the hydrogen acceptor by releasing the hydrogen under regeneration conditions.

12. The process as claimed in claim 11 wherein the catalyst and hydrogen acceptor are regenerated in a single regeneration vessel.

13. The process of claim 11 wherein the catalyst and hydrogen acceptor are regenerated in separate vessels

14. The process of claim 11 wherein the catalyst and hydrogen acceptor are each regenerated under different regeneration conditions

15. The process as claimed in claim 11 wherein the hydrogen released from the hydrogen acceptor is used for catalyst regeneration.

16. The process of claim 15 wherein supplemental hydrogen is supplied from an external source in order to properly complete the catalyst regeneration

17. The process of claim 11 wherein the hydrogen acceptor regeneration is accomplished under regeneration conditions including: feed rate, temperature and pressure that are substantially different from the aromatization conditions

18. The process of claim 11 wherein the hydrogen acceptor regeneration is accomplished with hydrogen containing offgas from the aromatization reaction

19. The process of claim 1 wherein the methane-containing gas stream is derived from biogas.

20. The process of claim 1 wherein the methane-containing gas stream is natural gas
